(19) United States
(12) Patent Application Publication
(10) Pub. No.: US 2023/0065561 A1
(43) Pub. Date: Mar. 2, 2023

(54) WEARABLE APPARATUS FOR THE TREATMENT OF MASTITIS

(71) Applicant: COSMOAESTHETICS PTY LTD, Kew (AU)

(72) Inventor: Masoud Tahghighi JAFARZADEH, Kew (AU)

(21) Appl. No.: 17/759,765

(22) PCT Filed: Feb. 1, 2021
(86) PCT No.: PCT/AU2021/050071

§ 371 Date: Jul. 29, 2022

(30) Foreign Application Priority Data
Jan. 31, 2020 (AU) 2020900260

(51) Int. Cl. A01J 7/04; A61B 5/01

(52) U.S. Cl. CPC A01J 7/04; A61B 5/015

(57) ABSTRACT

Wearable apparatus for the treatment of mastitis, the apparatus comprising a mammary gland wearable comprising an array of light source elements thereon emitting electromagnetic radiation having a wavelength in the range of 380-1050 nm, a power supply coupled to the array of light source elements, the wearable configured to be worn against a mammary gland in use wherein the array of light source elements emit the radiation which penetrates epidermis of the mammary gland for the treatment of mastitis.

### FIELD OF THE INVENTION

This invention relates generally to wearable apparatus for the treatment of mastitis. More particularly, this invention relates to wearable apparatus comprising an array of light source elements thereon emitting electromagnetic radiation having a wavelength in the range of 380-1050 nm.

### BACKGROUND OF THE INVENTION

Mastitis is bacterial inflammation of the breast, typically from Staphylococcus and Streptococci, and is usually associated with breastfeeding. Symptoms typically include local pain and redness and which may associated with fever and general soreness. Complications can include abscess formation. About 10% of breastfeeding women are affected.

Massage and application of heat prior to feeding can unblock milk ducts, although in more severe cases of mastitis heat or massage could worsen symptoms worse and cold compresses may therefore be better suited to reduce inflammation.

Treatment may involve steroid medications, antibiotics and even surgical procedures.

The present invention seeks to provide a way to treat mastitis, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a wearable apparatus for the treatment of mastitis. The apparatus comprises a mammary gland wearable comprising an array of light source elements thereon. The light source elements emit electromagnetic radiation having a wavelength in the range of 380-1050 nm which has bioactive effect for the treatment of mastitis.

The wearable is configured to be worn against a mammary gland in use wherein the array of light source elements emit the radiation which penetrates the epidermis of the mammary gland for the treatment of mastitis. Preferably, the light source elements have sufficient intensity to penetrate the epidermis with less than 10% attenuation.

For human application, the wearable may be worn around the breast, including discreetly under a bra or other similar clothing. In one form, the wearable comprises a semi-annular backing which may be folded conically to envelop the breast.

The user may control the operation of the wearable using a user interface electrically connected to the wearable via a supplied control cable. For example, the user may control the dosage applied according to the severity of infection.

The wearable may apply wavelength at different frequencies so that the wearable may, for example, treat inflammation and sensitivity and/or stimulate cell regeneration. Similarly, the user may control the user interface to control the type of wavelength applied depending on the type of therapy required.

The wearable may further comprise micro stimulation vibrators which induce micro vibrations under the epidermis of the breast thereby enhancing micro circulation and therefore healing. The micro stimulation vibrators may comprise piezoelectric vibrators which induce vibrations at less than 60 Hz, preferably less than 40 Hz.

The apparatus may control the wearable to target specific regions of the wearable depending on the nature, extent and location of the infection. In one form, the wearable comprises a touch sensitive backing which may be manipulated to control the dosage of radiation and/or micro stimulation applied at a particular region.

Alternatively, the wearable may be autosensing wherein the wearable comprises a plurality of temperature sensing elements across the surface of the wearable which may measure temperature at discrete locations across the breast. As such, the controller may target therapy to areas of increased temperature, indicative of infection, such as by controlling the dosage, wavelength and/or micro stimulation.

As such, with the foregoing in mind, in accordance with one embodiment, there is provided wearable apparatus for the treatment of mastitis, the apparatus comprising a mammary gland wearable comprising an array of light source elements thereon emitting electromagnetic radiation having a wavelength in the range of 380-1050 nm, a power supply coupled to the array of light source elements, the wearable configured to be worn against a mammary gland in use wherein the array of light source elements emit the radiation which penetrates epidermis of the mammary gland for the treatment of mastitis.

The intensity of the light source elements may penetrate the epidermis with less than 10% attenuation.

The apparatus may further may comprise a controller controlling the light source elements, wherein subsets of the light source elements or the light source elements may be independently controllable by the controller and wherein the controller controls the light source elements independently to target regions of the wearable.

The apparatus may further may comprise a controller controlling the light source elements, the controller having a user interface for selecting a mode of operation and wherein the controller controls the light source elements according to the mode of operation.

The controller may control at least one of intensity and operational duration of the light source elements according to the mode of operation.

The array of light source elements may comprise first and second arrays of light source elements emitting electromagnetic radiation at different wavelength ranges.

The wavelength ranges may comprise a wavelength range in the range of between approximately 410-600 nm.

The wavelength ranges may comprise a wavelength range in the range of between approximately 620-880 nm.

The apparatus may further may comprise a controller controlling the light source elements, the controller having a user interface for selecting a mode of operation and wherein the controller controls the first and second arrays of light source elements independently according to the mode of operation.

The wearable may comprise an array of temperature sensors which measures a temperature profile across the mammary gland in use.

The temperature sensors may be spaced apart from each other by less than 2 cm.

The controller may resolve sensed temperatures into a plurality of temperature bands.

The temperature bands may have a temperature range of approximately 3.5° C.,

The controller may control the array of light source elements to target areas of increased temperature.

The controller may apply a higher dosage of radiation to target the areas of increased temperature.

The array of light source elements may comprise first and second arrays of light source elements emitting electromagnetic radiation at first and second respective wavelength ranges and wherein the controller controls the first and second arrays of light source elements independently to apply either of the first and second respective wavelengths to target the areas of increased temperature.

The controller may store the areas of increased temperature for subsequent treatment.

The wearable may comprise a touch sensitive backing operably coupled to the controller to detect use touch interactions at certain regions thereof and wherein the controller controls the light source elements independently at a corresponding region of the wearable.

The wearable may comprise an array of micro stimulation vibrators which induces micro vibrations across the surface of the wearable.

The micro stimulation vibrators may comprise piezoelectric elements.

The piezoelectric elements may vibrate at less than 60 Hz.

The piezoelectric elements may vibrate at less than 40 Hz.

The apparatus may further may comprise a controller controlling the light source elements, wherein subsets of the micro stimulation vibrators or the micro stimulation vibrators may be independently controllable by the controller and wherein the controller controls the micro stimulation vibrators independently to target regions of the wearable.

The wearable may comprise a flexible backing with the plurality of light source elements located on a side thereof.

The backing may be non-transparent or translucent.

The wearable may comprise a transparent or translucent inner layer.

The wearable may comprise an inner adhesive layer.

The backing may be semi-annular comprising a radial cut out and central opening.

The backing may comprise an outer diameter of approximately 155 mm.

The backing may comprise an inner diameter of approximately 34 mm.

The wearable may comprise a plurality of petals each having respective light source elements thereon.

The petals may comprise PCB boards.

The petals may be spaced apart to allow radial channels therebetween allowing the backing to be folded conically.

The wearable may comprise an electrical connection socket for the attachment of a user interface via an electric cable.

The electrical connection socket may comprise power connections.

The socket may comprise positive and negative electrical contacts.

The electrical connection socket may comprise data connections.

The data connections may be serial data connections.

Serial data transmitted via the serial data contacts may control subsets of the light source elements.

The user interface may comprise a battery therein which supplies power to the wearable via the electrical cable.

The user interface may comprise a mode selector which selects a mode of operation.

The mode selected may controls the intensity and/or duration of the application of the light source elements to control the applied dosage.

The user interface may comprise operational indicators which indicate the time remaining of a mode of operation.

The wearable may comprise an array of temperature sensors and wherein the temperature sensors may be located between the light source elements.

The wearable may comprise an array of micro stimulation vibrators and wherein the micro stimulation vibrators may be located between the light source elements.

The wearable may comprise a radial arrangement of the light source elements thereon.

The light source elements may increase in density towards the centre of the wearable.

The array of light source elements may comprise first and second arrays of light source elements emitting electromagnetic radiation at different wavelength ranges and wherein the light source elements of the first and second arrays of light source elements may be arranged alternately.

The wearable may be for bovine application for the treatment of mastitis of milk cow udders which may comprise an udder pouch which may be worn over an udder in use and which may comprise the light source elements therein.

The pouch may hang loosely around the udder so as to not make direct contact therewith.

The pouch may comprise a plurality of teat apertures therethrough.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a wearable apparatus for the treatment of human breast mastitis;

FIG. 2 shows a side view of the wearable apparatus of FIG. 1;

FIG. 3 shows a plan view of the wearable of the apparatus of FIG. 1;

FIG. 4 shows a user interface for the apparatus;

FIG. 5 shows a side view of a wearable apparatus for the treatment of human breast mastitis in accordance with a further embodiment;

FIG. 6 shows a plan view of the wearable of FIG. 5;

FIG. 7 shows a plan view of the wearable of FIG. 3;

FIG. 8 shows a side view of the wearable;

FIG. 9 shows a functional control schematic of the apparatus in accordance with an embodiment;

FIG. 10 illustrates an exemplary method of operation of the apparatus;

FIG. 11 illustrates temperature band autosensing of the apparatus in accordance with an embodiment;

FIG. 12 shows a wearable apparatus for the treatment of bovine udder mastitis; and

FIG. 13 shows a perspective exploded representation of the wearable apparatus of FIG. 12.

### DESCRIPTION OF EMBODIMENTS

Wearable apparatus 100 for the treatment of mastitis comprises a mammary gland wearable 101 comprising an array of light source elements 102 thereon emitting electromagnetic radiation having a wavelength in the range of 380-1050 nm.

A power supply 108 is coupled to the array of light source elements 102. In use, the wearable 101 is configured to be worn against a mammary gland wherein the array of light source elements 102 emit the radiation which penetrates the epidermis of the mammary gland for the treatment of mastitis.

The light source elements 102 preferably have sufficient intensity to penetrate the epidermis (typically 70μ deep) with less than 10% attenuation.

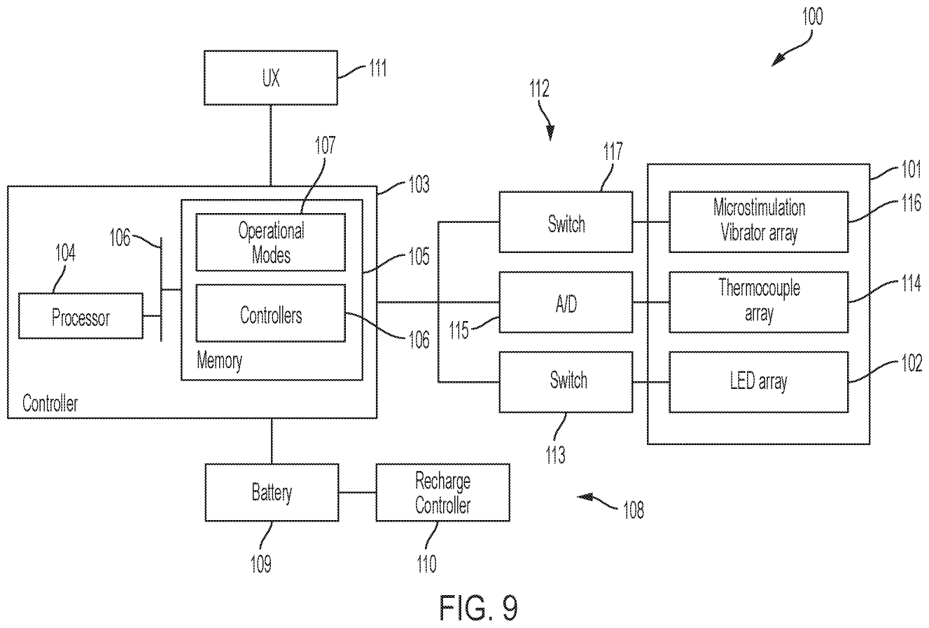

FIG. 9 shows a functional schematic of the apparatus 100 in accordance with an embodiment wherein the apparatus 100 comprises a controller 103 operably coupled to the wearable light source 101. The controller 103 may comprise a processor 104 in operable communication with a memory device 105 across a system bus 106.

The memory device 105 stores digital data including computer program code instructions. The computer program code instructions may be logically divided into a plurality of computer program code instruction controllers 106 for implementing the various functionality described herein. Furthermore, the data may comprise various operational modes 107.

In use, the processor 104 fetches these computer program code instructions and associated data from memory for 105 for interpretation and execution for implementing the functionality described herein. The apparatus 100 comprises a power supply 108 which may comprise a rechargeable battery 109 and a recharge controller 110 therefor.

The apparatus 100 may further comprise a user interface 111 for controlling the operation of the apparatus 100.

The controller 103 may comprise interfaces 112 for controlling the wearable light source 101. The apparatus 100 may comprise switching 113 which controls the array of light source elements 102.

In embodiments, the light source elements may comprise LEDs.

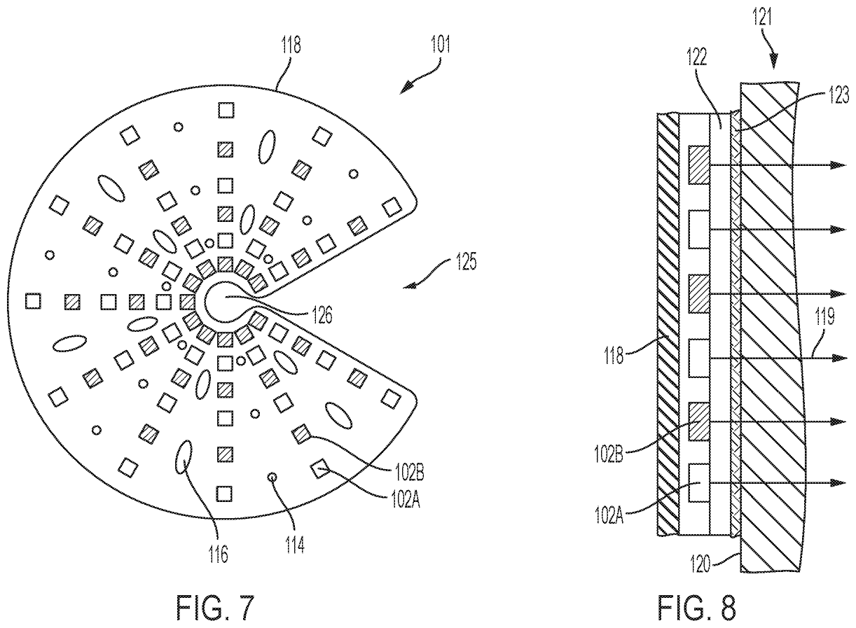

As shown in FIG. 7, the array of light source elements 102 may comprise a first array of light source elements 102A and a second set of light source elements 102B which emit radiation at the front wavelength.

In embodiment, the first array of light source elements 102A may emit more ultraviolet light such as in the range of 410-600 nm. Furthermore, the second array of light source elements 102B may emit more infrared light such as having a wavelength in the range of 620-880 nm.

The controller 103 may control the switching 113 to control each array 102A and 102B independently for treatment of mastitis with different wavelengths of light.

For example, the controller 103 may employ the first array of light source element 102A for addressing inflammation and sensitivity and the second array of light source elements 102B for promoting healing and stimulating cell growth and regeneration.

In embodiments, the wearable 101 comprises an array of temperature sensors 114 (such as thermocouples) which measures a temperature profile across the mammary gland. An analogue to digital interface 115 may interpret voltage generated by each thermocouple to interpret measure temperature. The temperature sensors 114 may be spaced across the wearable so as to be able to measure a temperature profile across the surface of the mammary gland, preferably to a resolution of less than 2 cm.

In embodiments, the wearable 101 comprises an array of microstimulation vibrators 116 and switching 117 therefore which induces micro vibrations across the surface of the wearable to stimulate micro circulation and therefore promote healing. The micro stimulators 116 may comprise piezoelectric elements which vibrate at less than 60 Hz, preferably less than 40 Hz.

The wearable 101 may comprise a flexible backing 118 with the plurality of light source elements 102 located on a side thereof to direct the radiation 119 through the epidermis 120 of the mammary gland 121. The backing 118 may be non-transparent or translucent to prevent or reduce radiation 102 escaping outwardly from the wearable 101. The wearable 101 may comprise a transparent or translucent inner layer 122. Furthermore, the wearable 101 may comprise an adhesive layer 123 for adhering the wearable to the epidermis 120.

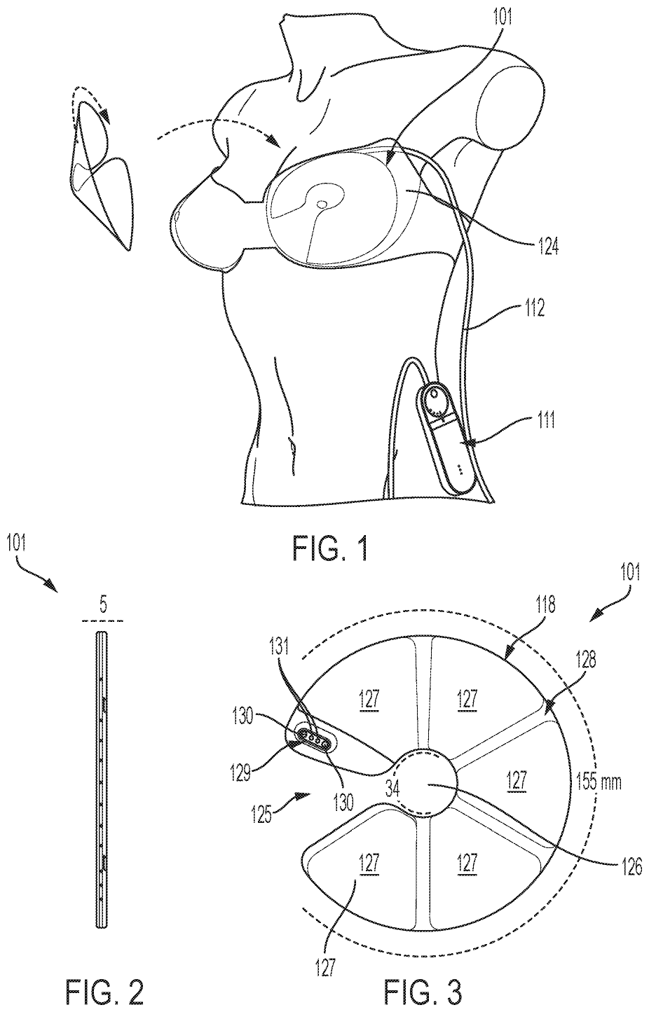

FIGS. 1-7 show an embodiment of the apparatus 100 for human application.

As shown in FIG. 7, the wearable 101 may be generally conforming in size and shape of the human breast 121 so as to be worn in the manner shown in FIG. 1, including discreetly under a bra 124 or clothing. In the embodiment shown in FIG. 1, the user is using one wearable 101 but, in embodiments, a wearable 101 may be used for each breast 121 simultaneously.

FIG. 7 shows an embodiment wherein the backing 118 is generally circular. More specifically, the backing 118 may be semi-annular comprising a radial cut out 125 and central opening 126. In this way, the wearable 101 may be laid flat in the manner shown in FIGS. 2 and 3 but folded conically in the manner shown in FIG. 1 to surround the breast 121 with the central opening 126 allowing the nipple of the breast 121 therethrough. The backing 118 may comprise an outer diameter of approximately 155 mm and an inner diameter of approximately 34 mm.

Referencing FIG. 7, the backing 118 may comprise a radial arrangement of the light source elements 102 thereon. As is shown, the light source elements 102 may increase in density towards the centre of the wearable 101 to thereby increase radiation dosage at or around the nipple. Furthermore, the first and second array of light source elements 102A and 102B may be arranged alternately so that the controller 103 may apply different wavelengths of radiation across the entire surface of the breast 121.

As is further shown, the array of temperature sensors 114 and micro stimulation vibrators 116 may be located between the light source elements 102.

As is shown in FIG. 3, the wearable 101 may comprise a plurality of petals 127 each having respective light source elements 102 thereon. The petals 127 may comprise nonflexible PCB boards but be spaced apart to allow radial channels 128 therebetween allowing the backing 118 to be folded conically.

An edge of the wearable 101 may comprise an electrical connection socket 129 for the attachment of the user interface 111 via an electric cable 112. The electrical connection socket 129 may be for power and data. Specifically, as shown in FIG. 3, the socket 129 may comprise positive and negative electrical contacts 130 and serial data contacts 131. Serial data transmitted via the serial data contacts 131 may control subsets of the light source elements 102, temperature sensors 114 and micro stimulation vibrators 116 allowing the controller 103 to target specific regions of the breast 121.

FIGS. 5 and 6 show an alternative embodiment wherein the wearable 101 is continually formed and which, as is shown in FIG. 5, may be convex so as to be ergonomically placed over the breast 121 without folding in the aforedescribed manner. The embodiment of FIGS. 5 and 6 may be approximately 54 millimetres in diameter.

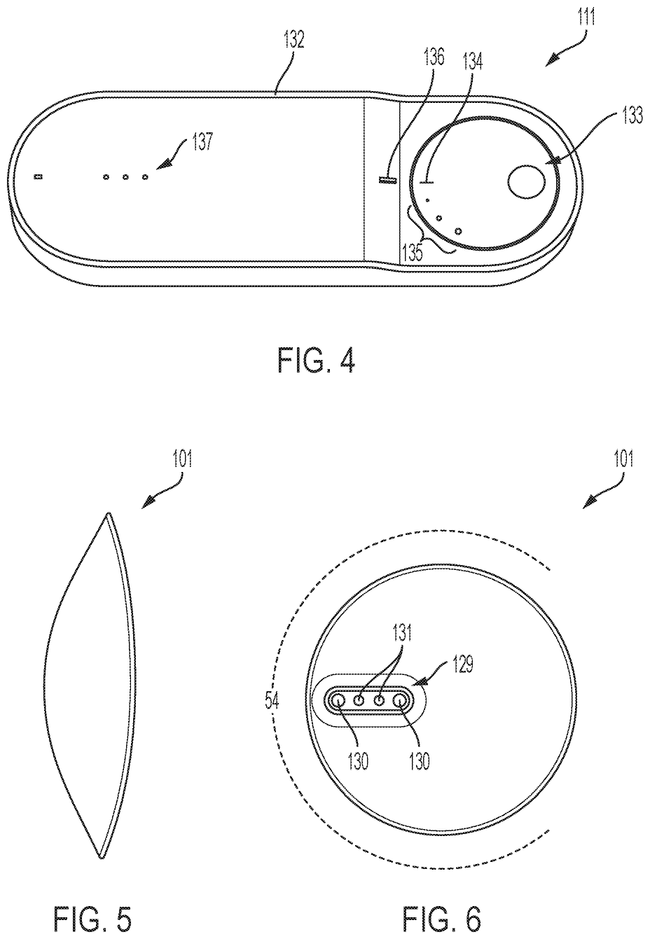

FIG. 4 shows a user interface 111 in accordance with an embodiment. As is shown in FIG. 1, the user interface 111 may be worn in a pocket, on a belt or the like and electrically connected to the wearable 101 via electrical and/or data cable 112.

User interface 111may comprise a generally elongate body 132. One end of the body 132 may comprise a rotary-type mode selector 133 which, when rotated selects a mode of operation.

In the embodiment shown, the selector 133 has an off indicator 134 and three operational mode indicators 135. The mode selected may control the intensity and/or duration of the application of the LEDs 102 to control the applied dosage. For example, the first mode may be a 4 J dosage program which lasts for 90 seconds, a second mode may be a 6 J dosage program which lasts for two minutes and a third mode may be a 10 J program which lasts three minutes.

The body 132 may comprise an audio port 136 for outputting audio indicative of the operation of the apparatus 100. The body 132 may further comprise operational indicators 137. In embodiments, the operational indicators 137 may indicate the time remaining of a program.

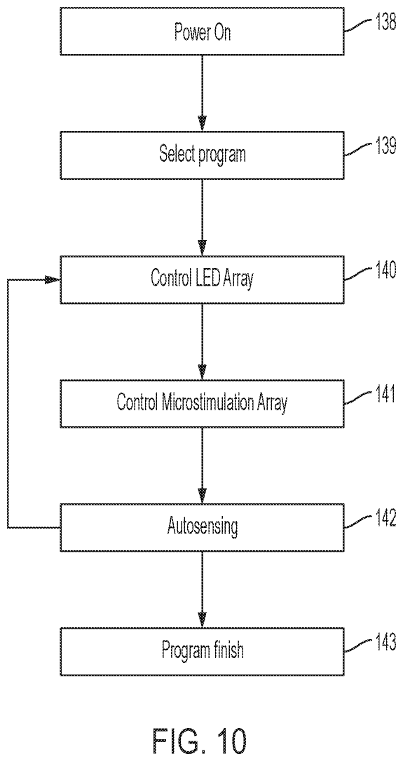

FIG. 10 illustrates exemplary utilisation of the apparatus 100 in accordance with an embodiment for the treatment of mastitis. A user would firstly place the wearable 101 over the affected breast 121 such as by folding the wearable 101 given in FIG. 3 conically so as to substantially surround the breast 121 in the manner shown in FIG. 1.

The wearable 101 may be worn discreetly beneath a bra 124 or alternatively adhered to the breast 121 using the adhesive layer 123.

The user interface 111 is electrically connected to the wearable using the supplied cable 112.

At step 138 the user powers on the apparatus 100. The user interface 111 may comprise the battery 109 therein which supplies power to the wearable 101 via the electrical cable 112.

At step 139, the user selects the program using the rotary program selector 133. As alluded to above, the selected program may control the amount of radiation dosage applied and which may be configured according to the severity of the infection.

Additionally, or alternatively, the program may control the wavelength of light applied by the wearable 101 such as whether inflammation and/or sensitivity therapy is required or cell regeneration stimulation as alluded to above.

At step 140 the controller 103 controls the array of light source elements 102 according to the selected program.

As alluded to above, the controller 103 may control the switching 113 to control the intensity and/or duration of operation of the light source elements 112. Furthermore, the controller 103 may control the switching 113 to control subsets of the light source elements to apply light at different wavelengths. The controller 103 may control the switching 113 to apply radiation across the entirety of the wearable 111 or alternatively to target specific regions of the wearable.

At step 141, the controller 131 may control the micro stimulation array to stimulate micro circulation across the entirety of the breast or for specific targeted regions thereof.

In embodiments, the wearable 101 comprises a touch sensitive backing operably coupled to the controller 103. The controller 103 may detect user touch interactions at certain regions of the touch sensitive backing. As such, the controller 103 may control the light source elements 102 at a corresponding region of the wearable. As such, if a particular region of the breast 121 is painful, the user may touch the touch sensitive backing at the painful region wherein the controller applies light at the corresponding location. The user may control the touch sensitive backing to apply different wavelengths and/or micro stimulation vibrations including of different frequencies.

Step 142 shows wherein the controller 113 uses the temperature sensor array 114 for autosensing a temperature profile across the breast 121.

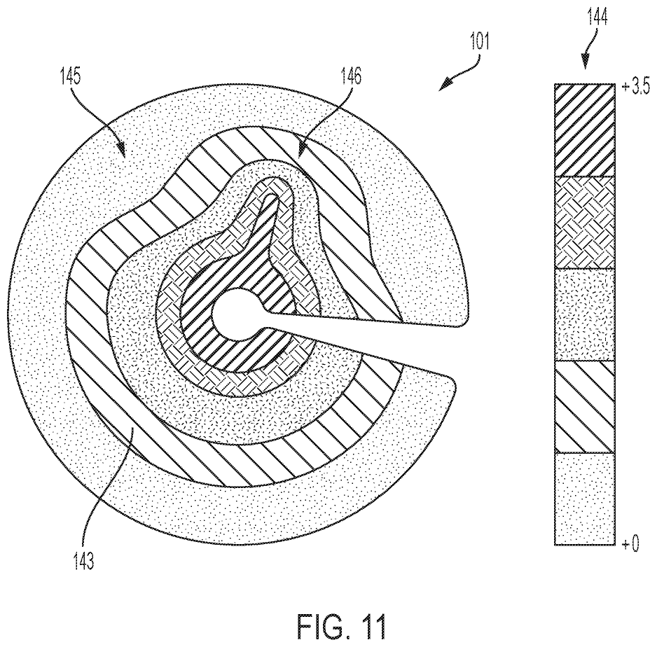

FIG. 11 shows an exemplary temperature profile 145 measured by the thermocouple array 114. The controller 103 may resolve the sensed temperatures into a plurality of temperature bands 143 which, according to the temperature scale 144 may vary by 3.5° C., the higher temperature indicating increased infection.

The exemplary profile 145 shown in FIG. 11 shows elevated temperature bands 143 surrounding the nipple indicative of increased infection at the nipple. Furthermore, the profile 145 shows elevated temperature radial aspect/region 146 indicative of a radial milk duct being infected.

As such, the controller 113 may control the array of light source elements 112 to target areas of increased temperature indicative of greater infection. For example, the controller 113 may apply a higher dosage of radiation to the region of the elevated temperature region 146.

Additionally, or alternatively, the controller 113 may control the subset arrays of the light source elements 112 to apply the wavelength of light addressing inflammation and sensitivity to the elevated temperature region 146.

In embodiments, the controller 103 may store regions of infection for subsequent treatment. For example, the controller 103 may subsequently apply a different wavelength of light encouraging cell regeneration.

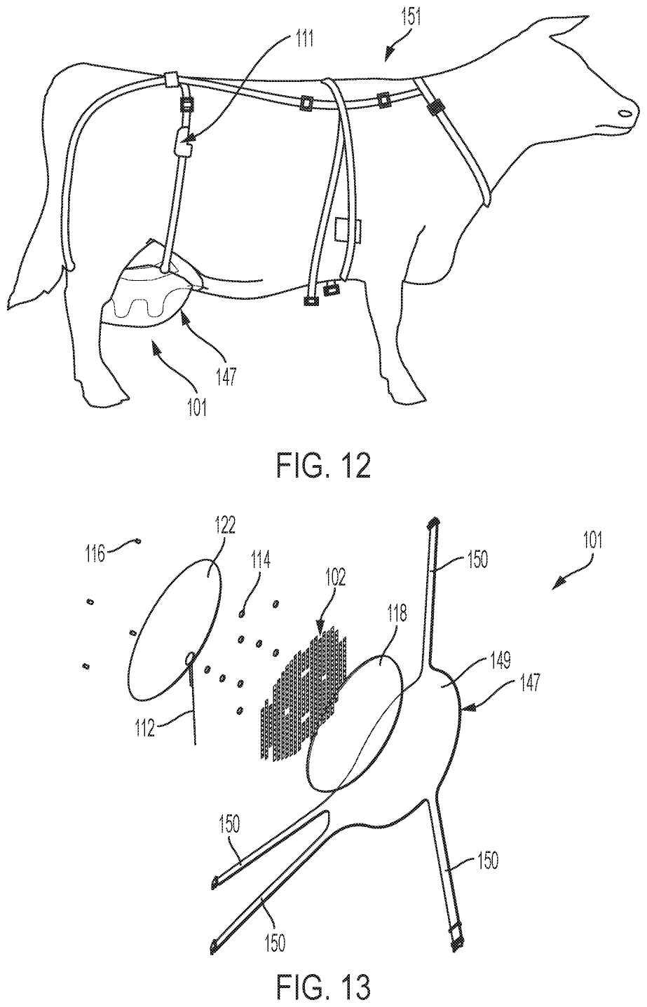

FIGS. 12 and 13 show an embodiment wherein the wearable 101 is for bovine application for the treatment of mastitis of milk cow udders.

In embodiment shown, the wearable comprises an udder pouch 147. In one form, the part 147 hangs loosely around the udder so as to not make direct contact therewith. In an alternative form, the pouch 147 comprises a plurality of teat apertures therethrough.

As shown in FIG. 11, the pouch 147 may comprise a central region 149 with radial straps 150. As shown in FIG. 12, the straps 150 may go around the hind legs of the animal. As further shown in FIG. 12, the user interface 111 may be supported by one of the straps 150. As yet further shown in FIG. 12, the animal may permanently wear a harness 151 to which the straps 150 may be attached when treatment is required.

In embodiments, a thermal imaging camera may be used to detect areas of elevated temperature of the mammary gland. For example, for bovine application, a thermal imaging camera operably coupled to a digital display, such as one of a mobile communication device, may display areas of increased temperature on the udder.

Information received from the thermal imaging camera by the controller 103 may be used to control the light source elements 102, including the wavelength range, operation, duration, intensity and targeted region. In embodiments, the pouch 147 may be of a material allowing infrared to pass therethrough so that thermal imaging of the under may be undertaken whilst the pouch 147 is been worn.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. Wearable apparatus for the treatment of mastitis, the apparatus comprising a mammary gland wearable comprising an array of light source elements thereon emitting electromagnetic radiation having a wavelength in the range of 380-1050 nm, a power supply coupled to the array of light source elements, the wearable configured to be worn against a mammary gland in use wherein the array of light source elements emit the radiation which penetrates epidermis of the mammary gland for the treatment of mastitis.

2. The apparatus as claimed in claim 1, wherein the intensity of the light source elements penetrates the epidermis with less than 10% attenuation.

3. The apparatus as claimed in claim 1, further comprising a controller controlling the light source elements, wherein subsets of the light source elements or the light source elements are independently controllable by the controller and wherein the controller controls the light source elements independently to target regions of the wearable.

4. The apparatus as claimed in claim 1, further comprising a controller controlling the light source elements, the controller having a user interface for selecting a mode of operation and wherein the controller controls the light source elements according to the mode of operation.

5. The apparatus as claimed in claim 4, wherein the controller controls at least one of intensity and operational duration of the light source elements according to the mode of operation.

6. The apparatus as claimed in claim 1, wherein the array of light source elements comprise first and second arrays of light source elements emitting electromagnetic radiation at different wavelength ranges.

7. The apparatus as claimed in claim 6, wherein the wavelength ranges comprise a wavelength range in the range of between approximately 410-600 nm.

8. The apparatus as claimed in claim 6, wherein the wavelength ranges comprise a wavelength range in the range of between approximately 620-880 nm.

9. The apparatus as claimed in claim 6, further comprising a controller controlling the light source elements, the controller having a user interface for selecting a mode of operation and wherein the controller controls the first and second arrays of light source elements independently according to the mode of operation.

10. The apparatus as claimed in claim 1, wherein the wearable comprises an array of temperature sensors which measures a temperature profile across the mammary gland in use.

11. The apparatus as claimed in claim 10, wherein the temperature sensors are spaced apart from each other by less than 2 cm.

12. The apparatus as claimed in claim 11, wherein the controller resolves sensed temperatures into a plurality of temperature bands.

13. The apparatus as claimed in claim 12, wherein the temperature bands have a temperature range of approximately 3.5° C.,

14. The apparatus as claimed in claim 10, wherein the controller controls the array of light source elements to target areas of increased temperature.

15. The apparatus as claimed in claim 14, wherein the controller applies a higher dosage of radiation to target the areas of increased temperature.

16. The apparatus as claimed in claim 14, wherein the array of light source elements comprise first and second arrays of light source elements emitting electromagnetic radiation at first and second respective wavelength ranges and wherein the controller controls the first and second arrays of light source elements independently to apply either of the first and second respective wavelengths to target the areas of increased temperature.

17. The apparatus as claimed in claim 14, wherein the controller stores the areas of increased temperature for subsequent treatment.

18. The apparatus as claimed in claim 1, wherein the wearable comprises a touch sensitive backing operably coupled to the controller to detect use touch interactions at certain regions thereof and wherein the controller controls the light source elements independently at a corresponding region of the wearable.

19. The apparatus as claimed in claim 1, wherein the wearable comprises an array of micro stimulation vibrators which induces micro vibrations across the surface of the wearable.

20. The apparatus as claimed in claim 19, wherein the micro stimulation vibrators comprise piezoelectric elements.

21. The apparatus as claimed in claim 19, wherein the piezoelectric elements vibrate at less than 60 Hz.

22. The apparatus as claimed in claim 19, wherein the piezoelectric elements vibrate at less than 40 Hz.

23. The apparatus as claimed in claim 1, further comprising a controller controlling the light source elements, wherein subsets of the micro stimulation vibrators or the micro stimulation vibrators are independently controllable by the controller and wherein the controller controls the micro stimulation vibrators independently to target regions of the wearable.

24. The apparatus as claimed in claim 1, wherein the wearable comprise a flexible backing with the plurality of light source elements located on a side thereof.

25. The apparatus as claimed in claim 24, wherein the backing is non-transparent or translucent.

26. The apparatus as claimed in claim 1, wherein the wearable comprises a transparent or translucent inner layer.

27. The apparatus as claimed in claim 1, wherein the wearable comprises an inner adhesive layer.

28. The apparatus as claimed in claim 24, wherein the backing is semi-annular comprising a radial cut out and central opening.

29. The apparatus as claimed in claim 24, wherein the backing comprises an outer diameter of approximately 155 mm.

30. The apparatus as claimed in claim 24, wherein the backing comprises an inner diameter of approximately 34 mm.

31. The apparatus as claimed in claim 28, wherein the wearable comprises a plurality of petals each having respective light source elements thereon.

32. The apparatus as claimed in claim 31, wherein the petals may comprise PCB boards.

33. The apparatus as claimed in claim 31, wherein the petals are spaced apart to allow radial channels therebetween allowing the backing to be folded conically.

34. The apparatus as claimed in claim 1, wherein the wearable comprises an electrical connection socket for the attachment of a user interface via an electric cable.

35. The apparatus as claimed in claim 34, wherein the electrical connection socket comprises power connections.

36. The apparatus as claimed in claim 35, wherein the socket comprises positive and negative electrical contacts.

37. The apparatus as claimed in claim 34, wherein the electrical connection socket comprises data connections.

38. The apparatus as claimed in claim 37, wherein the data connections are serial data connections.

39. The apparatus as claimed in claim 38, wherein serial data transmitted via the serial data contacts controls subsets of the light source elements.

40. The apparatus as claimed in claim 34, wherein the user interface comprises a battery therein which supplies power to the wearable via the electrical cable.

41. The apparatus as claimed in claim 34, wherein the user interface comprises a mode selector which selects a mode of operation.

42. The apparatus as claimed in claim 41, wherein the mode selected controls the intensity and/or duration of the application of the light source elements to control the applied dosage.

43. The apparatus as claimed in claim 34, wherein the user interface comprises operational indicators which indicate the time remaining of a mode of operation.

44. The apparatus as claimed in claim 1, wherein the wearable comprises an array of temperature sensors and wherein the temperature sensors are located between the light source elements.

45. The apparatus as claimed in claim 1, wherein the wearable comprises an array of micro stimulation vibrators and wherein the micro stimulation vibrators are located between the light source elements.

46. The apparatus as claimed in claim 1, wherein the wearable comprises a radial arrangement of the light source elements thereon.

47. The apparatus as claimed in claim 1, wherein the light source elements increase in density towards the centre of the wearable.

48. The apparatus as claimed in claim 1, wherein the array of light source elements comprise first and second arrays of light source elements emitting electromagnetic radiation at different wavelength ranges and wherein the light source elements of the first and second arrays of light source elements are arranged alternately.

49. The apparatus as claimed in claim 1, wherein the wearable is for bovine application for the treatment of mastitis of milk cow udders which comprises an udder pouch which is worn over an udder in use and which comprises the light source elements therein.

50. The apparatus as claimed in claim 49, wherein the pouch hangs loosely around the udder so as to not make direct contact therewith.

51. The apparatus as claimed in claim 50, wherein the pouch comprises a plurality of teat apertures therethrough.
